(19) United States
(12) Patent Application Publication
(10) Pub. No.: US 2010/0196660 A1
(43) Pub. Date: Aug. 5, 2010

(54) Process for the Production of Micro-Structured Construction Units by Means of Optical Lithography

(75) Inventor: MARTIN DRESSLER, BERLIN (DE)

Correspondence Address: LONDA, BRUCE S.;NORRIS MCLAUGHLIN & MARCUS, PA, 875 THIRD AVE, 8TH FLOOR, NEW YORK, NY, 10022, US

(73) Assignee: BAM Bundesanstalt fur Materialforschung und-prufung, Berlin (DE)

(21) Appl. No.: 12/699,171

(22) Filed: Feb. 3, 2010

(30) Foreign Application Priority Data
Feb. 5, 2009 (DE) 102009000642.7-54

(51) Int. Cl. G03F 7/20; B32B 3/10; B32B 3/26; B32B 3/30; A61F 2/02

(52) U.S. Cl. 428/138; 430/325; 430/324; 428/213; 428/189; 623/11.11

(57) ABSTRACT

The invention relates to a method for producing micro-structured shaped parts (22) and the resulting products, wherein a first negative or positive photosensitive layer (12) with a layer thickness (D1) is deposited on a substrate (10) and the first negative or positive photosensitive layer (12) is exposed area-by-area with light of a suitable wavelength; a second negative or positive photosensitive layer (12′) with a layer thickness (D2) is deposited on the first negative or positive photosensitive layer (12) and exposed area-by-area with light of a suitable wavelength, wherein the exposed regions (16′) in the second layer (12′) are identical to and/or different from the exposed regions (16) in the first layer (12); the steps of depositing and exposing the photosensitive layer (12, 12′) area-by-area are performed repeatedly until a predetermined height H is attained, wherein the exposed regions (16, 16′) of the photosensitive layers (12, 12′) represent the positive or negative of the microstructure of the shaped part (22); the exposed (16, 16′) or the unexposed regions (18, 18′) of the shaped part (22) are washed out with a developer; and the remaining preform (20) is subsequently solidified.

The invention relates to a method for producing micro-structured shaped parts with a photolithographic process. The invention also relates to micro-structured shaped parts produced with this process.

Components with defined and complex interior structures, for example undercuts or apertures, can be produced in sizes from several centimeters to the submillimeter range (about 100 μm). Although it is possible (i) to provide surfaces with still finer structures and (ii) to introduce smaller structures with defined dimensions in components, only the surfaces are accessible in the first case while it is not possible in the second case to manipulate the size and, more importantly, the position of each structural element. One example is here the use of agents to form pores in ceramic materials. Although micrometer-sized pores can be produced, the additional control required to position each individual structural element, i.e., each individual pore, has so far not been possible. Such precisely controlled generation of structures has hitherto only been possible down to the submillimeter range.

During recent years and as a result of the limitations presented by conventional production methods, rapid prototyping methods have been established in addition to the conventional mechanical machining by so-called subtractive methods (material-removing methods) like drilling, milling, turning. Rapid prototyping operates additively, i.e., a desired structure is not produced by removing excess material, but instead by intentionally adding material. Rapid prototyping has allowed the production of more complex structures than was possible with conventional subtractive methods.

A large number of different methods and combinations of methods can be used in the context of rapid prototyping. Most common is either (a) the direct additive formation of components using powder-based techniques, or (b) creation of a mold using photopolymer-based methods. For different reasons, these two approaches have in common that they do not allow the fabrication of structures smaller than down to the submillimeter range.

With the conventional subtractive methods, the resolution that can be produced down to the submillimeter range is limited by the size of the tools. Additive rapid prototyping process absolutely require in the case (a) that the employed powder has excellent flow characteristics and hence a comparatively large minimum particle size. This requirement also significantly limits the possible grain size of the powder and hence also the attainable resolution. In the case (b) of the additive rapid prototyping process, the resolution is limited by the attainable focal spot size of the laser beam used for the local polymerization, the penetration depth into the polymer bath, i.e., into the monomer solution, as well as the wetting ability and flowability of the solid suspension during subsequent shape infiltration.

The manufacturing principle employed with the conventional rapid prototyping processes can be subdivided as follows: UV hardening methods (stereo lithography—SL), sintering methods (selective laser sintering—SLS), lamination methods (laminated object manufacturing—LOM), extrusion methods (fused deposition molding—FDM), and 3-D-printing methods (3-D-printing).

With SL, a laser beam scans the surface of a photosensitive liquid. At the locations irradiated by the laser beam, a chemical reaction starts which causes local hardening of the polymer. By lowering the plane that was already scanned with the laser beam into the polymer bath and by renewed irradiation, a component is produced layer-by-layer.

With SLS, the laser beam scans a powder fill instead of the surface of a polymer bath. The laser power is selected such that the powder bed is strongly heated locally, with the powder particles sintering together at the irradiated location. Like with SL, the plane in which a structure has already been inscribed is lowered and a new layer of powder is applied.

In LOM, a laser is also used for structuring. Several layers of a foil are stacked on top of one another, i.e., laminated. By locally cutting the foil with the laser, the laser defines the contour of the desired component after each lamination step.

In the extrusion method FDM, a paste-like strand of a mixture of solid and plasticizers is pressed out of a fine nozzle and placed on a support while the nozzle performs a controlled motion in the x-y direction. After the first layer is produced, additional layers are produced by placing the new strand on the already placed strand parts.

3-D-printing is a method where the contour of the component is not defined by a laser, but by a fine fluid jet. A binder is applied in a powder layer with a fine jet. A coating knife pushes a new powder layer over the previous layer, and the printing of a binder pattern is repeated.

This method known from rapid prototyping can attain the following resolutions: SL [symbol] 100 μm, SLS [symbol] 76 μm, LOM [symbol] 51 μm, FDM [symbol] 254 μm und 3D-Printing [symbol] 178 μm (Yan und Gu, A review of rapid prototyping technologies and systems, Computer-Aided Design 28 (1996) 307-318).

The limiting factors are predominantly caused by the following parameters. With SL, the size of the laser spot and the minimum penetration depth of the laser beam into the polymer bath are critical. The resolution decreases with increasing laser spot size and penetration depth into the polymer bath. With SLS, the size of the laser spot is also important, with the flow characteristics of the employed powder having greater impact. All powder-based methods have in common that the maximum attainable spatial resolution is many times greater than the particle size of the employed powder. The particle size can also not be arbitrarily reduced, because powder particles in a size range of several micrometers do no longer have a satisfactory flow characteristic. This represents the major problem with 3-D-printing. With LOM, the thickness of the employed laminate limits the resolution. The resolution of the FDM method is limited by the nozzle width of the micro-extruder.

Two additional problems common with all methods for rapid prototyping are a requirement for support structures and the ability to remove excess material from components with cavities.

Support structures are required, for example, when producing components with undercurrents using FTM and SL, because the components are surrounded only by air (FDM) or a polymer bath (SL). None of the powder-based methods (SLS, 3-D-printing) and LOM requires support structures, because the surrounding powder bed (SLS, 3-D-printing) or laminate (LOM) serve as support.

Excess material can be most easily removed from the printed preform in the SL method. The liquid polymer that has not yet been irradiated by the laser beam can simply be drained from the printed body. With the powder-based methods (SLS, 3-D-printing), removal of the excess powder is more difficult due to the limited flowability of solid fills. Most difficult is the removal of excess material in LOM, where the excess material is located in a cavity not in form of a liquid or powder fill, but as a solid block of laminated foil.

A microstructure with improved resolution, i.e., with a resolution in the micrometer and sub-micrometer range, is particularly important in the field of biological sciences. Animal cells are embedded in their physiological surroundings in three-dimensional micro-structured networks, with their development and behavior affected by interaction with their immediate environment. To optimize otherwise artificial laboratory conditions, for example for tissue engineering or improving implant materials, three-dimensional biocompatible micro-structured materials and shaped parts on a sub-cellular scale are required.

It is therefore an object of the present invention to obviate the disadvantages of the prior art and to provide a method for micro-structuring shaped parts with improved resolution.

It is another object of the invention to provide three-dimensional micro-structured shaped parts with a microstructure in the sub-micrometer range.

The object is attained with the method and the shaped part of the independent claims. Advantageous embodiments are recited in the dependent claims.

The invention relates to a method for producing micro-structured shaped parts, wherein a first negative or positive photosensitive layer with a layer thickness is deposited on a substrate and the first negative or positive photosensitive layer is exposed area-by-area with light of a suitable wavelengths; a second negative or positive photosensitive layer with a layer thickness is deposited on the first negative or positive photosensitive layer and exposed area-by-area with light of a suitable wavelengths, wherein the exposed regions in the second layer are identical to and/or different from the exposed regions in the first layer; the steps of depositing and exposing of the photosensitive layer area-by-area are performed repeatedly until a predetermined height H is attained, wherein the exposed regions of the photosensitive layers represent the positive or negative of the microstructure of the shaped part; the exposed or the unexposed regions of the shaped part are washed out with a developer; and the remaining preform is subsequently solidified.

According to the invention, at least two thin layers are deposited on the substrate. The total number of layers is defined by the height of the component to be produced and the layer thickness. The layer thickness of each applied layer is between 100 nm to several micrometers and is selected so as not to limit the attainable resolution of the microstructure. A selective exposure with an exposure mask takes place after each deposition step. After a sufficient number of layers is deposited and exposed, i.e., when the desired height of the component is attained, a photochemical treatment follows which selectively washes out all exposed locations. The micro-structured “preform” produced in this manner is subsequently solidified by an additional, preferably physical, treatment.

The method of the invention makes it possible to produce complex components with structures defined by their size and position. Advantageously, unlike with conventional subtractive methods and known rapid prototyping methods, shaped parts with substantially smaller structural dimensions on a micrometer and sub-micrometer scale can be provided. This approach advantageously solves the problem associated with the geometric resolution of conventional methods that are limited to the submillimeter range. This improved resolution opens new possibilities not only for structuring implants, but also for applications such as structured coatings.

For example, the method of the invention can advantageously be used for intentionally controlling the size and position of each structural element in the component. This position control, in addition to improvements of the spatial resolution, is particularly important, for example, for ceramic components.

The resolution according to the invention can only be attained by combining lithographic techniques and the principles of rapid prototyping according to the invention. The method of the invention is characterized in that additional lithographically structured surfaces can be applied to a surface that has previously been structured by lithographic techniques. Advantageously, lithographic methods provide high resolution which is transferred to three-dimensional shaped bodies with the method of the invention.

Because the photosensitive layers are only developed at the end of the building process of the microstructure, support structures can be eliminated with the invention. In addition, removing the excess material does not pose a problem, because the regions of the photosensitive layers that are not required are removed in liquid form during the development step. This is also an essential prerequisite for three-dimensional microstructures in the micrometer and sub-micrometer range.

The micro-structured preform produced after development is subsequently solidified, preferably by using thermal solidification processes. The preform is preferably solidified by sintering. According to the invention, other methods can also be used for final solidification, for example renewed irradiation which is longer than the exposure, or irradiation with high energy. The selection of a suitable method for final solidification depends, in particular, on the materials used for the photosensitive layers.

In a preferred embodiment of the invention, the photosensitive layer is a suspension of a negative or a positive photoresist and solid particles suspended therein. Accordingly, the photoresist serves according to the invention as a binder matrix to hold the applied ceramic particles in place. In this way, a layer in the micrometer and sub-micrometer range can be produced. With the additive method, the attainable resolution limit of the microstructures is essentially determined by the layer thickness of the individual layers. At the same time, introducing solid particles provides excellent stability which is required, for example, for bio-ceramics.

According to the invention, all known conventional negative or positive photoresist can be used. A negative resist polymerizes through exposure and subsequent heating for stability, i.e., the exposed regions remain in place following development. With positive resists, the already solidified resist becomes again soluble for suitable developer solutions following exposure, meaning that after development only those regions remain that are protected from the irradiation by a mask and are therefore not exposed. According to the invention, both types of photoresist can be used; however, a particular microstructure is always constructed from a single type of resist.

Preferably, microstructures are built up from layers of photoresist, which are biocompatible and inert against physiological solutions and environmental conditions after hardening. According to the invention, suitable photoresists are photoresists of the AZ®- and the TI-photoresist-family distributed by MicroChemicals®, for example AZ® 6612, AZ® 9260, AZ® 1505, AZ® 1512HS, AZ® 1514H, AZ® 1518, AZ® MiR 701, AZ® ECI 3027, AZ® 9260, AZ® nLOF 2000, AZ® 5214 E, TI Spray, TI 35E/ES or of the series ma-P or ma-N distributed by Micro Resist Technology GmbH, for example ma-P 1200, ma-N 400, ma-N 1400, ma-N 2400, mr-UVL 6000 or EpoCore and EpoClad. A particularly preferred photoresist is the ma-P 1215-photoresist.

The particle size of the solid particles determines the minimal layer thickness of the individual layers. According to the invention, the particles are suspended in the photoresist so as to prevent the formation of agglomerates or particle accumulations. Conventional methods, such as ultrasound or vortex formation as well as chemical dispersion agents, are used for forming the suspensions. According to the invention, the solid particles are uniformly distributed in the photoresist and are present as individual particles.

In an advantageous embodiment of the invention, the average diameter of the solid particles is 100 nm to 10 μm, preferably 100 nm to 5 μm, and more preferably 100 nm to 3 μm. Particles with a smaller average particle size, preferably in a range from 20 nm to 100 nm, can also be used with a method of the invention. According to the invention, the lower limit for the particle size and hence also for the resolution is determined by the dispersibility of the particles in the photoresist.

All solid particles that can be produced with the corresponding sizes are suitable for the method of the present invention. In an advantageous embodiment of the invention, the employed solid particles may be plastic particles, metal particles, ceramic particles or mixtures thereof. More particularly, hydroxyl apatite, tricalcium phosphate, or particles comprised of the bone replacement material described in DE 102 49 627 (claim 1) are used. Preferably, Ca₂K₁₋ₓNa₁₊ₓ(PO₄)₂ particles, wherein x=0-0.9, and more preferably Ca₂KNa(PO₄)₂ particles according to DE 102 49 627 or pure Ca₂KNa(PO₄)₂ particles are used.

The solid content of the suspension of photoresist and the solid particles may be varied according to the invention. A higher solid content is used when the micro-structured shaped part is subjected to high mechanical loading and requires a greater hardness. A smaller solid content is an advantageous when a particularly high resolution is to be attained. To produce the required thin layers, the suspensions advantageously have low viscosity.

According to an advantageous embodiment, the solid content of the suspension of photoresist and solid particles is in a range from 1 to 90 weight-%, preferably from 10 to 70 weight-%, and more preferably from 10 to 50 weight-%, based on the total weight of the mixture. In a particularly preferred embodiment of the invention, the solid content is about 30 weight-%, based on the total weight of the mixture.

In particular, the layer thickness is determined by the viscosity of the suspension. According to the invention, suitable suspensions have a viscosity from 1 to 60 mPa, preferably from 5 to 50 mPa, and more preferably from 10 to 30 mPa. The suspensions are deposited according to the invention by rotational methods, such as spin coating. The desired layer thickness is defined by selecting the rotation speed and the viscosity of the suspension. According to the invention, the layer thicknesses D1, D2, . . . Dn−1, Dn of the photosensitive layers (12, 12′) are in a range from 100 nm to 10 μm, preferably in a range from 100 nm to 5 μm, and more preferably in a range from 100 nm to 3 μm, wherein n is a positive integer.

According to the invention, the first photosensitive layer is deposited on a substrate. A substrate in the present invention is defined as any suitable surface on which a micro-structured shaped body can be constructed. Preferably, flat and smooth substrates are used. The substrates according to the invention are chemically inert against the solvent of the photosensitive layer. A preferred substrate is glass. In a particularly preferred embodiment of the invention, the substrate itself is a shaped body, on the surface of which the three-dimensional micro-structured shaped body is built up. Three-dimensional substrates consist preferably of metallic, ceramic or polymer materials, such as Ti₆Al₄V, Al₂O₃ or polymethyl methacrylate (PMMA).

In an advantageous embodiment of the invention, the substrate remains on the micro-structured shaped body at the end of the process. Alternatively, the substrate can be removed from the micro-structured shaped body at the end of the process. According to the invention, the substrate itself is dissolved and/or removed from the microstructure. Suitable methods for removing the substrate are etching or dissolution. If the substrate is to be removed from the micro-structured shaped body, then the surface of the substrate, on which the microstructure is deposited, is preferably provided with an anti-adhesion layer. Suitable anti-adhesion layers are known to the skilled artisan.

According to the invention, the photosensitive layer is exposed with electromagnetic radiation. The wavelength of the radiation is determined by the absorption characteristic of the photosensitive components of the employed photoresist. Preferably, radiation in the range of visible light or UV radiation is used for exposure. According to the invention, the photosensitive layer is exposed not entirely, but only area-by-area. The exposure is performed either by using a lamp in conjunction with a photomask or point-wise with a laser. Depending on the type of resist used, either the areas of the photosensitive layer are exposed that are to be removed, or the areas that should remain. The exposure time depends on the concentration and the absorption characteristic of the solid particles and of the photoresist, the illumination power of the radiation source and the layer thickness.

According to the invention, the exposure of the photosensitive layer is advantageously selected so that only the photosensitive material of one layer is exposed with the selected layer thickness. This is preferably achieved by varying the irradiation intensity and/or the duration of the exposure. Suitable means for controlling the irradiation intensity and/or the exposure duration, for example shutters or neutral filters/gray filters, are known to persons skilled in the art.

In an advantageous embodiment of the invention, photoresists with different properties are used in adjoining photosensitive layers. The photosensitivity as well as the other properties of the resists, such as viscosity or solvent, can be varied. According to the invention, for example resists of different viscosity are used for varying the layer thickness of adjoining layers. Resists based on different solvents are preferably used when otherwise a portion of a subjacent layer would be dissolved when the next layer is deposited, which would result in an imprecise structure. A variation of the photosensitive properties of the resists is used with the invention, for example, to prevent additional exposure of the subjacent layers.

In another advantageous embodiment of the invention, a protective layer is deposited between the photosensitive layers. The protective layer has a layer thickness Ds in a range from 100 nm to 10 μm, preferably in a range from 100 nm to 5 μm, and more preferably in a range from 100 nm to 3 μm. Preferably, the thickness of the protective layer is selected to be identical to or smaller than the thickness of the photosensitive layer. Preferably, the protective layer is not chemically resistant to the developer for the photoresists. In this way, the protective layer is protected by the photoresist in those regions where the photoresist remains, and is removed by the developer in the other regions. During the development step, the microstructure is then transferred to the protective layer. In another embodiment of the invention, the protective layer can be chemically inert to the developer. In this case, the protective layer is physically unstable against the final solidification process, for example heat treatment. For example, the protective layer can then be burned out to produce the structure of the protective layer. In a preferred embodiment, the protective layer is chemically stable, i.e., resistant against the solvents of the photoresists. The protective layer can either be additionally resistant to the sintering process or can be burned out during sintering.

For example, natural and/or synthetic hydro-colloids and/or gelling agents, such as gelatine, pectin, agar and/or mixtures thereof, can be used as the protective layer. Preferably, the gelatine is dissolved in polar solvents, in particular water. The protective layer according to the invention has a viscosity in a range from 1 to 60 mPa, preferably in a range from 5 to 50 mPa, and more preferably in a range from 10 to 40 mPa. When using a gelatine solution, the viscosity of the solution can be adjusted by way of the gelatine content and/or the temperature of application. Preferably, gelatine solutions with a gelatine concentration from 0.1 to 50 weight-%, preferably with a concentration from 1 to 30 weight-%, and more preferably with a concentration from 5 to 20 weight-% are used. The most preferred solution has a gelatine concentration of about 10 weight-%. According to the invention, the layer thickness of the protective layer can also be varied by the deposition temperature, with the viscosity of the solution decreasing with increasing temperature. The protective layer according to the invention is preferably used in a temperature range from 30 to 90° C., preferably in a temperature range from 40 to 80° C., more preferably in a temperature range from 50 to 70° C., in particular at about 60° C.

In another preferred embodiment of the invention, the protective layer contains a material capable of absorbing radiation of the wavelength used for exposing the photosensitive layer. Preferably, materials capable of absorbing and/or reflecting visible and/or UV light are used. Suitable materials, such as dyes, color pigments, benzotriazole, triazine or bezophenone are known to persons skilled in the art.

It is a further object of the invention to provide a micro-structured shaped part with at least two structured layers, which is produced with the method of the invention. Preferably, a three-dimensional shaped part is produced with the method of the invention, wherein the entire structure of the shaped part is on a micrometer scale and/or sub-micrometer scale. The three-dimensional micro-structured shaped part of the invention consists of at least 2 to n layers, wherein n is a positive integer.

In a preferred embodiment of the invention, the lateral and/or vertical dimension of the structural elements of the micro-structures is in a range from 100 nm to 10 μm, preferably from 100 nm to 5 μm, and more preferably from 100 nm to 3 μm. In this way, a fully structured shaped part according to the invention is produced with microstructures having a continuous resolution on a micrometer and/or sub-micrometer scale. In particular, the structural elements of the microstructure according to the invention can be undercuts and/or apertures.

In an advantageous embodiment, the micro-structured shaped part has an overall height H from 10 to 500 μm, preferably from 50 to 200 μm, more preferably of about 100 μm. The height H represents the overall height of the three-dimensional micro-structured shaped part. Preferably, the height H corresponds to the sum of the layer thicknesses D1, D2, . . . Dn−1, Dn of the photosensitive layers (ΣD1, D2, . . . Dn−1, Dn), or of the sum of the photosensitive layers D1, D2, . . . Dn−1, Dn and number of protective layers Ds (ΣD1, D2, . . . Dn−1, Dn (n×Ds)).

In an alternative embodiment, larger micro-structured shaped parts can also be produced with the method of the invention. The size of the micro-structured shaped parts is essentially unrestricted with this method, only the required time increases with increasing size.

In a particularly preferred embodiment of the invention, the micro-structured shaped part is an implant and/or a three-dimensional micro-array or a similar surface. The three-dimensional micro-structured shaped part of the invention is hence either a solid body or a partial body which preferably surrounds and/or at least partially covers another shaped body. Particularly preferred micro-structured shaped parts are bio-ceramics, substrates for cell assays and/or micro-arrays.

Exemplary embodiments of the invention will now be described with reference to the appended figures.

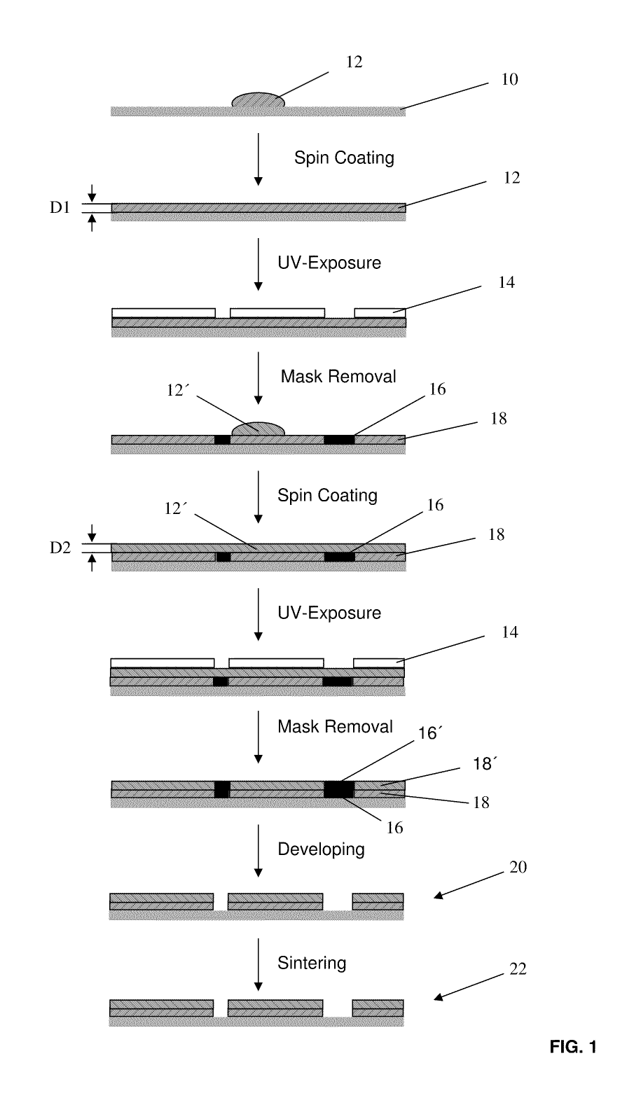

FIG. 1 shows a diagram of the method of the invention for producing a micro-structured shaped part; and

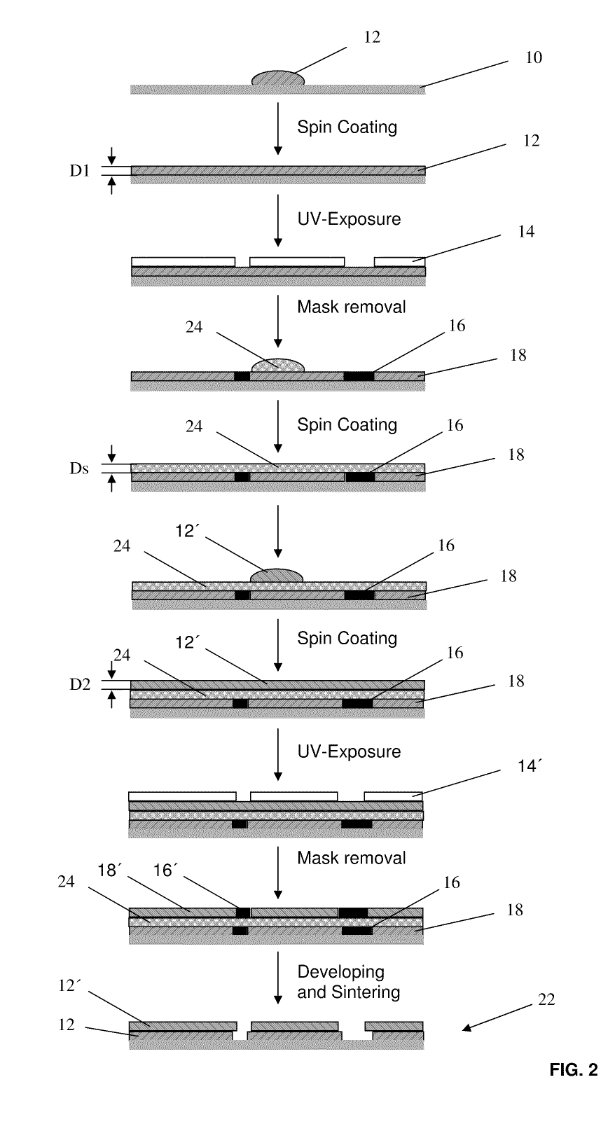

FIG. 2 shows a diagram of the method of the invention for producing a micro-structured shaped part by using a protective layer.

### EXAMPLE 1

FIG. 1 shows schematically the method of the invention for producing a micro-structured shaped part 22. A photosensitive layer 12 with a layer thickness D1 is deposited on a substrate 10 by spin coating. The photosensitive layer 12 is then exposed area-by-area through a photomask 14, so that after exposure, the photosensitive layer 12 is divided into exposed regions 16 and unexposed regions 18. As a result of the exposure, the photosensitive layer 12 is either sensitive or insensitive to the developer. The photomask 14 is then removed and a second photosensitive layer 12′ with a layer thickness D2 is deposited on the first, already exposed photosensitive layer 12, and again exposed through the photomask 14. In the present example, the same photomask 14 is used for both exposures, so that the exposed and unexposed regions 16, 16′ and 18, 18′ in the layers 12 and 12′ correspond to one another. After exposure, the photosensitive layers 12, 12′ are developed. In this example where a positive photoresist is used, the exposed regions are removed during development. The preform 20 produced by the development is subsequently solidified by sintering, which produces the finished micro-structured shaped part 22. In a modification of the illustrated embodiment, more than two layers 12, 12′ can be deposited and developed in order to attain a greater height H of the shaped part.

### EXAMPLE 2

FIG. 2 shows schematically the method of the invention for producing a micro-structured shaped part 22 by using a protective layer 24. A first photosensitive layer 12 is deposited on the substrate 10 by spin coating and exposed area-by-area through the photomask 14, producing exposed regions 16 and unexposed regions 18. The protective layer 24, which is resistant against the solvents of the photoresists of the photosensitive layers 12, 12′, is then deposited. The second photosensitive layer 12′ is then deposited on the protective layer 24 and also exposed through a second photomask 14′, producing the exposed regions 16′ and the unexposed regions 18′. Different photomask 14, 14′ are used to expose the two photosensitive layers 12, 12′. The photosensitive layers 12, 12′ are developed after exposure, whereby the exposed regions 16, 16′ are removed. The preform 20 is then solidified by sintering. During sintering, the protective layer 24 is burned out, causing the photosensitive layers 12, 12′ to collapse. Accordingly, the completed micro-structured shaped part 22 is also only composed of the photosensitive layers 12, 12′ after sintering.

### EXAMPLE 3

For producing a suspension according to the invention, ceramic Ca₂KNa(PO₄)₂ particles (according to DE 102 49 627) with a diameter of d₅₀=3 μm were suspended in a UV-sensitive photoresist. Two suspensions of photoresist were prepared with different solvents. ma-P 1215 photoresist was used for the first suspension and AZ-Mir 701 photoresist for the second suspension. The suspensions were homogenized on the vortexer for 10 minutes to uniformly distribute the introduced ceramic particles and to dissolve any generated agglomerates. The solid concentration in both suspensions was 30 weight-%. The first suspension was then deposited on a glass substrate by spin coating at 4000 RPM and exposed through a photomask for 15 minutes with light from a UV lamp (6-10 mW/cm²) having a wavelength of 300-460 nm. The generated photosensitive layer had a layer thickness D1=3 μm. Thereafter, the second suspension was deposited on the first photosensitive layer by spin coating at 4000 RPM (D2=3 μm) and exposed with light of the same wavelength. This process was repeated with the first suspension and then again with the second suspension, whereby the layers made of the first and second suspension are alternatingly arranged. All four layers were exposed with the same photomask. The comparatively long exposure time does not pose a fundamental obstacle for using the method of the invention, because UV lamps or lasers with higher power are commercially available. After all layers are deposited and exposed in designated regions, the unexposed photoresist is selectively washed out with developer solvents. After the developer liquid is selective washed out, the pattern of the employed photomask was imaged.

### EXAMPLE 4

Another microstructure was prepared, with a protective layer disposed between two corresponding photosensitive layers. A suspension of ma-P 1215-photoresist and Ca₂KNa(PO₄)₂ particles (according to DE 102 49 627) with d₅₀=3 μm, as described in example 3, was prepared for the photosensitive layer. A 10% solution of a gelatine (Merck) heated to 60° C. was used, which produces very uniform thin films. Gelatine has the important property of being insoluble in acetone, which is the solvent for the photoresist. Three photosensitive layers (layer thickness 3 μm) and three protective layers (layer thickness 3 μm) were alternatingly deposited by spin coating at 4000 RPM and exposed area-by-area with a photomask. After the deposition, the unexposed photoresist is selectively washed out with the developer solution ma-D 331 (Micro Resist Technology). After the developer liquid was selectively washed out, an image of the pattern of the employed photomask was produced. The gelatine layer disposed between the photosensitive layers can completely prevent a partial dissolution of the photoresist layers. The overall height of the micro-structured shaped part corresponds to the sum of the individual layer thicknesses of the deposited layers. The component was subsequently sintered. With the sintering process, the protective layers are burned out and the microstructure collapses to the height of the photosensitive layers.

### EXAMPLE 5

A micro-structured shaped part as in Example 4 was produced. 1% hydroxyl-phenyl-benzotriazol was added to the protective layer to reliably prevent unintended UV exposure of photoresist layers underneath the newly deposited photoresist layer, without requiring precise control of the power of the UV lamp and of the exposure time.

### LIST OF REFERENCES SYMBOLS

10 Substrate
12, 12′ Photosensitive layer
14, 14′ Photomask
16, 16′ Exposed region
18, 18′ Unexposed region
20 Preform
22 Micro-structured shaped part
24 Protective layer
D1, D2 Layer thickness photosensitive layer
Ds Layer thickness protective layer

1. Method for producing micro-structured shaped parts (22), wherein a first negative or positive photosensitive layer (12) with a layer thickness (D1) is deposited on a substrate (10) and the first negative or positive photosensitive layer (12) is exposed area-by-area with light of a suitable wavelength,
a second negative or positive photosensitive layer (12′) with a layer thickness (D2) is deposited on the first negative or positive photosensitive layer (12) and exposed area-by-area with light of a suitable wavelength, wherein the exposed regions (16′) in the second layer (12′) are identical to and/or different from the exposed regions (16) in the first layer (12);
the steps of depositing and exposing the photosensitive layer (12, 12′) area-by-area are repeatedly performed until a predetermined height (H) is attained, wherein the exposed regions (16, 16′) of the photosensitive layers (12, 12′) represent the positive or negative of the microstructure of the shaped part (22);
the exposed (16, 16′) or the unexposed regions (18, 18′) of the shaped part (22) are washed out with a developer;
and the remaining preform (20) is subsequently solidified.

2. Method according to claim 1, wherein the photosensitive layer (12, 12′) is a suspension of a negative or positive photoresist and solid particles suspended therein.

3. Method according to claim 2, wherein the average diameter of the solid particles is 100 nm to 10 μm, preferably 100 nm to 5 μm, and more preferably 100 nm to 3 μm.

4. Method according to claim 2, wherein the solid particles are plastic particles, metal particles, ceramic particles or mixtures thereof, preferably hydroxyl apatite, tricalcium phosphate, Ca₂K₁₋ₓNa₁₊ₓ(PO₄)₂ particles, wherein x=0-0.9, or Ca₂KNa(PO₄)₂ particles.

5. Method according to claim 2, wherein the solid content of the suspension made of photoresist and solid particles is in a range from 1 to 90 weight-%, preferably from 10 to 50 weight-%, and more preferably about 30 weight-%, based on the total weight of the mixture.

6. Method according to claim 2, wherein the layer thickness (D1, D2) of the photosensitive layer (12, 12′) is in a range from 100 nm to 10 μm, preferably in a range from 100 nm to 5 μm, and more preferably in a range from 100 nm to 3 μm.

7. Method according to claim 2, wherein photoresist with different viscosity and/or based on different solvents is used in adjoining photosensitive layers (12, 12′).

8. Method according to claim 2, wherein a protective layer (24) is deposited between the photosensitive layers (12, 12′).

9. Method according to claim 8, wherein the protective layer (24) is resistant against the solvents of the photoresists.

10. Method according to claim 8, wherein the protective layer (24) is a gelling agent solution, preferably a gelatine solution.

11. Method according to claim 8, wherein the protective layer (24) comprises a substance suitable to absorb light of the wavelength used for exposing the photosensitive layer (12, 12′).

12. Method according to claim 8, wherein the photosensitive layer (12, 12′) is exposed with UV light.

13. Method according to claim 8, wherein the preform (20) is sintered for final solidification.

14. Micro-structured shaped part (22) comprising at least two structured layers (12, 12′), produced with a method according to claim 1.

15. Micro-structured shaped part (22) according to claim 14, wherein the lateral and/or vertical dimension of the structural elements of the micro-structures is in a range from 100 nm to 10 μm, preferably from 100 nm to 5 μm, and more preferably from 100 nm to 3 μm.

16. Micro-structured shaped part (22) according to claim 14, wherein the structural elements are undercuts and/or apertures.

17. Micro-structured shaped part (22) according to claim 14, wherein the shaped part (22) is an implant and/or a three-dimensional micro-array or a similar surface.
